(19) United States
(12) Patent Application Publication
(10) Pub. No.: US 2018/0117495 A1
(43) Pub. Date: May 3, 2018

(54) METHOD FOR THE CONTINUOUS ELUTION OF A PRODUCT FROM CHROMATOGRAPHY COLUMNS

(71) Applicant: BAYER AKTIENGESELLSCHAFT, LEVERKUSEN (DE)

(72) Inventors: PETER SCHWAN, LEVERKUSEN (DE); KERSTIN BAUMARTH, WUPPERTAL (DE); MARTIN LOBEDANN, KÖLN (DE)

(21) Appl. No.: 15/573,059

(22) PCT Filed: May 10, 2016
(86) PCT No.: PCT/EP2016/060368

§ 371 Date: Nov. 9, 2017

(30) Foreign Application Priority Data
May 13, 2015 (EP) 15167518.8

(51) Int. Cl. B01D 15/16; B01D 15/18; B01D 15/42

(52) U.S. Cl. CPC B01D 15/16; B01D 15/3804; B01D 15/424; B01D 15/1871

(57) ABSTRACT

The invention provides a method for the continuous elution of a biopharmaceutical, biological, macromolecular product from more than one chromatography column, comprising the steps of:

(a) providing a product stream via an inlet,

(b) simultaneously loading n front chromatography columns with the product stream in a loading zone with a loading time t_{B}, an outlet stream of the n front chromatography columns being simultaneously distributed to at least n-1 recovery chromatography columns in a recovery zone,

where n is between 1 and 5, and

where the n front chromatography columns at a given time point have a varying loading time t_{B} between 0 and L₁, between L₁ and L₂, and up to between Lₙ₋₁ and Lₙ,

where each front chromatography column has a total loading time of Lₙ,

characterized in that, periodically after a constant switch time t_{S} of Lₙ₋₁, the front chromatography column n comes out of the loading zone and a recovery chromatography column from the recovery zone enters the loading zone,

(c) washing the product-loaded column from step (b) with at least one wash buffer,

(d) eluting the product from the washed column from step (c) with an elution time t_{E}, which is ≥80% of the switch time t_{S}, and

that, over 80% of the total run time t_{G}, at least one chromatography column is continuously in elution step (d).

The invention relates to a method for the continuous elution of a product from chromatography columns.

In biotechnological production, proteins are usually purified in batches. This means that the individual production cycles are handled discontinuously in a batchwise manner, with the entire product being removed after completion of a production cycle. To carry out production again, it is then necessary to start a new separate product cycle or batch.

In recent years, it has been increasingly demonstrated that a continuous procedure can also be performed in biotechnological production, where the process runs without interruptions, in contrast to a batch process.

The highly regulated pharmaceutical production requires great effort in terms of time, technology and personnel to provide cleaned and sterilized bioreactors and to ensure a sterile product. To reliably avoid cross-contamination in the event of a product changeover in a multipurpose system or between two product batches, what is required apart from cleaning is a very complex cleaning validation, which, if applicable, must be repeated in the event of a method adaptation.

This applies both to upstream processing (USP), i.e. the production of biological products in fermenters, and to downstream processing (DSP), i.e. the purification of the fermentation products.

Especially in the case of fermentation, a sterile environment is essential for a successful culture. To sterilize batch fermenters or fed-batch fermenters, the SIP technique (SIP=sterilization-in-place) is generally used.

The downtime of reactors resulting from the necessary cleaning and sterilization procedures can take up a significant share of reactor availability, especially in the case of short usage periods and frequent product changes. This affects, for example, the method steps of media preparation and fermentation in USP of biotechnological production, and solubilization, freezing, thawing, pH adjustment, production separation, e.g. via chromatography, precipitation or crystallization, adjusting buffers and virus inactivation in DSP.

In the downstream method, the regulatory requirements are a microbe-reduced method management. Therefore, there is no need for a sterile method in the case of batch operation. However, in a continuous method, the purification of the protein is performed over a relatively long period of time if possible without cleaning steps. This preferably occurs without sterilization steps during the purification. This is the case even though the risk of microbial contamination is many times higher than in the case of a simple batch operation.

WO2012/078677 describes a method and a system for the continuous processing of biopharmaceutical products by means of chromatography and the integration thereof in a production system, more particularly in a disposable system. Although WO2012/078677 provides approaches for the continuous production of biopharmaceutical and biological products, the disclosed solution is not adequate in practice. WO2012/078677 also does not disclose the continuous elution of a product.

US 2014/0255994 A1 discloses an integrated continuous method for producing therapeutic proteins. However, US 2014/0255994 A1 does not disclose the feature that it is possible to carry out elution continuously in such a method.

EP 2 182 990 A1 discloses a method for sterilizing chromatography columns by using hot water vapour.

First of all, some terms will be defined in more detail.

In the context of the invention, a continuous method, or continuous elution, means any method for carrying out at least two method steps in series, the outlet stream of an upstream step being conveyed to a downstream step in said method. The downstream step starts the processing of the product stream before the upstream step has been completed. Typically, in a continuous method, part of the product stream is always being conveyed in the production system and is referred to as a “continuous product stream”. Accordingly, a continuous conveyance or transfer of a product stream from an upstream unit to a downstream unit means that the downstream unit is already operating before the upstream unit is put out of operation, i.e. that two successively connected units simultaneously method the product stream flowing through them.

In the context of the invention, the term “front chromatography column” means a chromatography column which, in a series connection, is in the first position during loading and is directly loaded with product stream.

In the context of the invention, the term “recovery chromatography column” means a chromatography column which, in a series connection, is in the second position after the front chromatography columns during loading and is loaded with the product stream from the front chromatography columns.

In the context of the invention, the “loading time t_{B}” means the time in which a chromatography column as front chromatography column is situated in the loading zone.

In the context of the invention, the “switch time t_{S}” means that, periodically after a constant switch time t_{S}, the front chromatography column n comes out of the loading zone and a recovery chromatography column from the recovery zone enters the loading zone.

In the context of the invention, the “elution time t_{E}” means the time in which the product is eluted by means of an elution buffer from a chromatography column into the product outlet.

In the context of the invention, the “total run time t_{G}” means the time during which the method according to the invention is operated altogether without interruption.

In the context of the invention, the term “microbe-reduced” means a state of reduced microbial count, i.e. a microorganism count per unit area or unit volume of virtually zero, which is achievable by a suitable microbe-reduction method, such as gamma irradiation, beta irradiation, autoclaving, ethylene oxide (ETO) treatment and “steam-in-place” (SIP) treatment.

In the context of the invention, the term “disposable article” means that the parts in question that come into contact with product, more particularly apparatuses, tanks, filters and connecting elements, are suitable for one-time use with subsequent disposal, it being possible for said tanks to be made both from plastic and from metal. In the context of the invention, the term also encompasses reusable articles, made of steel for instance, which are used only once in the method according to the invention and are then no longer used in the method. In the context of the invention, said reusable articles, made of steel for example, are then also referred to as objects “used as disposable articles”. Such disposable articles that are used can then also be referred to as “single-use” articles (“SU technology”) in the method according to the invention. These yet further improve the microbe-reduced state of the method according to the invention and of a modular system.

In the context of the invention, the term “product stream” means the cell-free fluid from a heterogeneous cell culture-fluid mixture containing the product, and the result of each of the other method steps of the method according to the invention, i.e. the product stream after filtration, after chromatography, after virus depletion, after ultrafiltration, after diafiltration, or after further steps of the method according to the invention. Said product streams may have different concentrations and different degrees of purity.

In the context of the invention, the term “virus depletion” means a reduction in the concentration of active viruses per unit volume of the fluid to be treated, right up to complete inactivation and/or removal of the viruses present in the fluid to be treated.

In the context of the invention, the term “bubble trap” means a device for collecting gas bubbles, with the fluid in question being degassed when this is taking place.

In the context of the invention, the term “modular” means that the individual steps of the method according to the invention can be carried out in separate modules that are connected to one another, the modules being preconfigured and microbe-reduced and it being possible to connect them to one another in a closed manner and in different combinations.

In the context of the invention, the term “modular system” means a series of modules (“units”) connected to one another for carrying out at least two downstream and/or upstream steps, in which a fluid (“product stream”) can be conveyed. According to the invention, the units are suitable for continuously carrying out a step and can be operated with a continuous fluid stream (“product stream”). The individual modules of the “modular system” can be connected to one another in any combination.

In the context of the invention, the term “closed” means the mode of operation of the method according to the invention and of the modular system according to the invention, which are operated such that the product produced and/or processed by said method and said modular system is not exposed to the room environment. Materials, objects, buffers and the like can be added from the outside to the closed method according to the invention and the corresponding closed modular system according to the invention, though this addition takes place in such a way that an exposure of the produced and/or processed product to the room environment is avoided.

The customary methods known in the prior art have a range of disadvantages, which will be dealt with below.

Known methods for producing biopharmaceutical and biological products typically comprise the following production steps, which are connected to one another:
1. perfusion culture
2. cell retention system,

as an alternative to steps 1 and 2, a feed-batch culture can also be envisaged,
3. cell removal
4. buffer or media exchange, preferably with concentration
5. bioburden reduction, preferably by sterile filtration
6. capture chromatography.

Typically, further steps are carried out for further purification of the product stream, more particularly:
7. virus inactivation
8. neutralization, and
9. optionally a further depth filtration, bioburden reduction (sterile filtration).

In view of the high quality standards in the production of biopharmaceuticals, the following steps are typically additionally carried out:
10. chromatographic intermediate and high-quality purification
11. bioburden reduction, for example sterile filtration
12. virus filtration
13. buffer exchange and preferably concentration, and
14. sterile filtration.

In the above-described production, cells in a fermenter containing nutrient solution produce a biological product, for instance a protein, for example a therapeutic protein. The used nutrient solution is also an ideal growth medium for microorganisms, such as bacteria and spores. As this growth of such microorganisms is not desired a problem arises from these circumstances. Said undesired growth of microorganisms especially becomes a problem in the case of relatively long run times because the nutrient solution becomes increasingly contaminated as the run time of the process increases, right up to an exponential growth of microorganisms and thus a total loss of the batch of the biological product that is produced.

To cope with the demand for a rapid and flexible reloading of the production system while maintaining maximum cleanliness and sterility, concepts for a continuous production, preferably using disposable technology, are attracting a constantly growing interest in the market.

For relatively long run times of such a process, ranging from two or more hours over days to weeks, customary sanitization measures are, however, insufficient, for example the customary “clean-in-place” (CIP) measures, such as sanitization by means of 1 M NaOH for example. In the case of run times above two or more hours, such customary processes and systems therefore have the disadvantage that they are highly susceptible to possible contamination and/or possible microbial growth.

Generally, when producing monoclonal antibodies for example, the first chromatography step using a protein A column is followed by a virus inactivation by means of an acidic pH <4 or an alkaline pH>9. This step is time-critical, i.e. for the purpose of virus inactivation, a certain time, generally <2 h, may not be fallen short of. However, in the case of an excessively long residence time, for example 4 hours, the antibody will be destroyed.

Therefore, there is a need for a method for the continuous purification of a product from a heterogeneous cell culture-fluid mixture, which due to its microbe-reduced state allows a continuous mode of operation for several weeks.

In this regard, the continuous virus inactivation coupled with an upstream chromatography step represents a special challenge, since the elution of the antibody is always done batchwise in discrete time steps with fluctuations in concentration and pH.

It is therefore an object of the present invention to develop a method and a corresponding system, by means of which a product, for instance a protein, can be continuously eluted from a product stream over a period of several hours right up several weeks. The continuous elution stream is continuously depleted of viruses.

The invention achieves this object by providing a method for the continuous elution of a biopharmaceutical, biological, macromolecular product from more than one chromatography column, comprising the steps of:

(a) providing a product stream via an inlet,

(b) simultaneously loading n front chromatography columns with the product stream in a loading zone with a loading time t_{B}, an outlet stream of the n front chromatography columns being simultaneously distributed to at least n-1 recovery chromatography columns in a recovery zone,

where n is between 1 and 5, and

where the n front chromatography columns at a given time point have a varying loading time t_{B} between 0 and L₁, between L₁ and L₂, and up to between Lₙ₋₁ and Lₙ,

where each front chromatography column has a total loading time of Lₙ,

characterized in that, periodically after a constant switch time t_{S} of Lₙ-Lₙ₋₁, the front chromatography column n comes out of the loading zone and a recovery chromatography column from the recovery zone enters the loading zone,

(c) washing the product-loaded column from step (b) with at least one wash buffer,

(d) eluting the product from the washed column from step (c) with an elution time t_{E}, which is ≥80% of the switch time t_{S}, and

that, over 80% of the total run time t_{G}, at least one chromatography column is continuously in elution step (d).

The technical advantage of such a continuous elution is that, when producing biopharmaceutical, biological, macromolecular products that are sensitive, e.g. monoclonal antibodies, the residence times of the product in the particular method step are minimized This is the case because, typically, a first chromatography step using, for example, a protein A column is followed by a virus inactivation by means of an acidic pH <4 or an alkaline pH>9. This step is time-critical, i.e. for the purpose of virus inactivation, a certain time, generally <2 h, may not be fallen short of. However, in the case of an excessively long residence time, for example 4 hours, the antibody will be destroyed.

Preferably, in the method according to the invention after elution step (d), the eluted product is mixed by means of a homogenization step (e), preferably by means of a recycling loop with a recycling tank, or by means of a recycling loop without a recycling tank, or by means of a single-use static mixer.

In a further embodiment of the method according to the invention, the method further comprises the step (f) regenerating the eluted column from step (d).

The front chromatography columns and recovery chromatography columns that are used in the method according to the invention can exhibit any suitable binding principle, for instance affinity of the product for a ligand, ionic interactions, metal chelate binding, hydrophobic interactions or pure van der Waals forces.

Preferably, the front chromatography columns and the recovery chromatography columns bind product in accordance with the principle of affinity, via ionic interactions, via metal chelate binding, via hydrophobic interactions or via van der Waals forces. In the case of binding in accordance with the principle of affinity the front chromatography columns and the recovery chromatography columns in the case comprise a ligand preferably selected from the group consisting of protein A, protein G, protein L, IgM, IgG and a recombinant protein which is different from protein A, protein G, protein L, IgM and IgG and which has an affinity for the product.

Preferably, the biopharmaceutical, biological macromolecular product comprises a protein, peptide or a DNA or RNA, the protein or peptide being selected from the group consisting of monoclonal antibodies, polyclonal antibodies, recombinant proteins and protein vaccines, and the DNA or RNA being part of a DNA and/or RNA active ingredient or vaccine.

In a further embodiment of the method according to the invention, the pH of the eluted product from step (d) is additionally adjusted by means of a pH adjuster, preferably before homogenization step (e), or during homogenization step (e).

Preferably, the homogenized product from step (e) runs through a defined residence-time passage, preferably a coiled flow inverter (CFI).

Preferably, the total run time t_{G} of the method is at least 4 hours, preferably at least 8 hours, preferably at least 12 hours, preferably at least 24 hours, more preferably at least 48 hours, more preferably at least 7 days, more preferably at least 4 weeks, and particularly preferred at least 8 weeks. Such a long run time of several weeks in a preferably continuous mode of operation is enabled by a closed, modular and, especially preferred, microbe-reduced mode of operation of the method.

Preferably, steps (a) to (d), preferably steps (a) to (f), are carried out in a microbe-reduced manner Preferably all the used elements that come into contact with the product, more particularly the front chromatography columns and the recovery chromatography columns, are subjected to microbe reduction by a suitable microbe-reduction method.

Such suitable microbe-reduction methods can be selected from the group consisting of gamma irradiation, beta irradiation, autoclaving, ethylene oxide (ETO) treatment, ozone treatment (O₃), hydrogen peroxide treatment (H₂O₂) and steam-in-place (SIP) treatment.

Preferably, all the liquids, gases and solids that are used in the method are subjected to microbe reduction, the microbe reduction preferably being achieved by means of a filtration through a filter having a pore size of preferably ≤0.45 μm. In-process sterilization is preferably not carried out during the method.

In a further embodiment of the invention, a degassing of all fluids which come onto the chromatography column is carried out before step (b), the degassing being achieved preferably by means of at least one bubble trap and/or by means of at least one hydrophobic microfiltration membrane via vacuum and/or by treatment with ultrasound and/or by sparging with helium.

The present invention including preferred embodiments will be elucidated in conjunction with the following drawings and examples without being restricted thereto. The embodiments can be combined with one another as desired, if the contrary is not clearly evident from the context.

The following are shown:

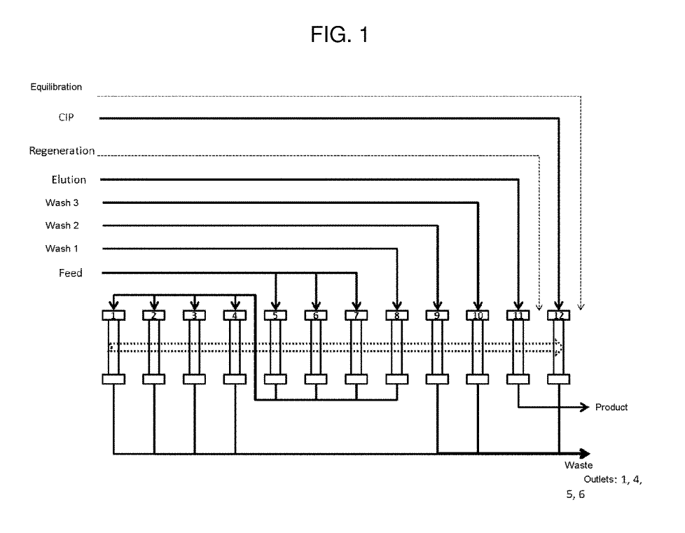

FIG. 1: Schematic layout of the inlet streams and of the outlet streams in a continuous chromatography system having 12 columns The inlet streams are configured as in Example 1. In the chromatography cycle, the columns move from left to right, i.e. from the first loading in the recovery zone up to CIP and equilibration. Moreover, it is shown that individual method steps such as, for example, regeneration and equilibration cannot be continuous.

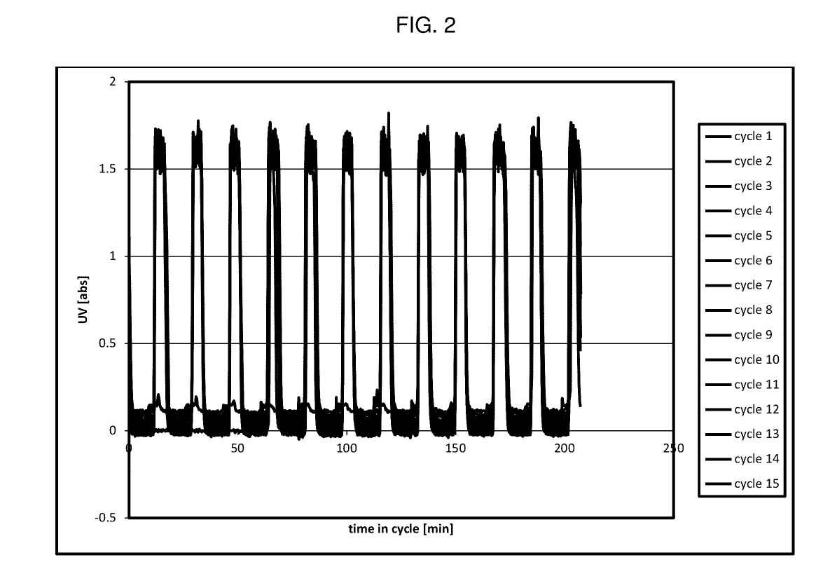

FIG. 2: UV absorption at 280 nm of the elution peaks during 15 cycles from Example 1.

The UV signal is representative of the concentration of the proteins in the elution. The elution stream is constant, but the protein concentration is periodical.

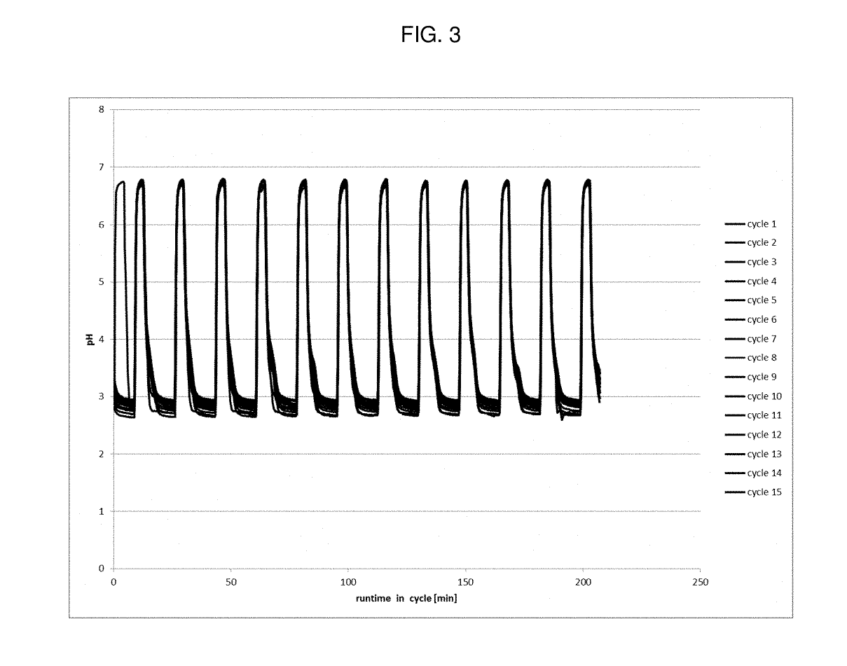

FIG. 3: pH of the elution peaks during 15 cycles from Example 1. The elution stream is constant, but the pH fluctuates in a periodical manner, since the Wash 3 buffer must first be displaced.

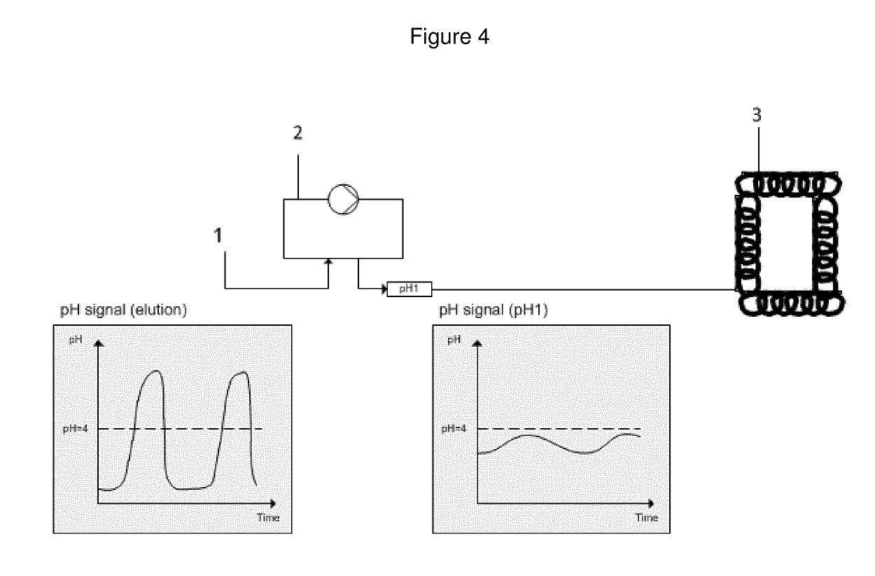

FIG. 4: The principle of continuous elution with subsequent continuous virus inactivation. In this case, the fluctuating continuous elution stream is mixed by means of a homogenization loop and then runs continuously to the residence-time passage.

1 Continuous elution stream with pH fluctuation

2 Homogenization loop

3 CFI (cold flow inverter) residence-time passage

### EXAMPLE 1

In Example 1, an IgG1 monoclonal antibody was used. The fermenter broth was prepared via a fed-batch method. The feed concentration corresponded to 0.8-1.5 g/l. The used chromatography system was a Tarpon system. In said system, 12 columns having an inner diameter of 16 mm were used. The columns were packed with the protein A resin Mabselect Sure from GE. The columns were packed according to manufacturer's specifications to a height of 8 cm with a volume of 16.1 ml. The Tarpon system had 8 inlets. The configuration of the inlets was as follows:

Inlet 1: Connection of the outlet of the front columns

Inlet 2: Fermenter broth feed

Inlet 3: Wash 1 buffer

Inlet 4: Wash 2 buffer

Inlet 5: Elution buffer

Inlet 6: Regeneration buffer

Inlet 7: Cleaning in place (CIP)

Inlet 8: Equilibration buffer or Wash 3 buffer

The outlets of the chromatography system were as follows.

Outlet 1: Waste stream

Outlet 2: Closed

Outlet 3: Elution

Outlet 4: Wash waste

Outlet 5: Flow-through waste from the recovery zone

Outlet 6: Outlet of the front columns connected to Inlet 1

Inlets 2-6 were each provided with the respective solutions/buffers via separate piston pumps.

The buffer systems were composed as follows:

Wash 1: 50 mM Tris, 2.5 M NaCl, pH 7

Wash 2: 50 mM NaAc, 1 M NaCl, pH 5

Elution buffer: 100 mM Na acetate, 50 mM NaCl, pH 5

Regeneration buffer: 50 mM Na acetate, 500 mM NaCl, pH 2.7

Wash 3/equilibration buffer: 50 mM Tris/50 mM NaCl, pH 7

CIP: 0.5 M NaOH

In the loading zone, each front column was loaded with 32.25 column volumes (CV) at a feed rate of 30 ml/min During this loading, three columns were always simultaneously in the loading zone, whereas 4 recovery columns at a time were situated in the recovery zone. These recovery columns could collect unbound IgG from the loading zone. The switch time was 17.29 min

After a front column was loaded for 3 switch times, said column was washed in Wash 1 within one switch time with 10 CV. Thereafter, the column was likewise washed within one switch time with 10 CV in Wash 2. Thereafter, Wash 3 reduced the salt load on the washed column, within half a switch time with 5 CV.

The washed column was subsequently eluted with 4.5 CV in exactly one switch time, whereby a continuous elution stream at ˜4.2 ml/min was realized during the entire process. Regeneration, CIP and equilibration of the column were then each carried out within half a switch time with a volume of 5, 3 and 5 CV.

The continuous elution stream was fed at a flow rate of 4.2 ml/min to a homogenization loop. In this feed, the pH in the eluate fluctuated from 3.1 to 6.6, as shown schematically in FIG. 4. For the virus inactivation, a pH<4 was required in order to achieve an effective virus inactivation. The strong pH fluctuations were reduced to a maximum pH of 3.9 as a result of the homogenization, as shown in FIG. 4. The homogenization loop consisted of a length of tubing having an inner diameter of 6.4 mm and a volume of 30 ml. In said loop, a peristaltic pump pumped the contents at a flow rate of 380 ml/min The risk of a short-circuit flow was minimized by the direction of flow running against the direction from inlet to outlet. The homogenized product flowed with the same flow rate as the eluate from the homogenization loop. Said product was then guided into the residence-time loop. The residence-time loop was a coiled flow inverter (CFI) having a narrow residence-time distribution comparable with that of an ideal plug flow. The minimum residence time in the CFI was 60 mM The setup of the CFI is described in Klutz et al. (Klutz, S., Kurt, S. K., Lobedann, M., Kockmann, N., 2015) and in “Narrow residence time distribution in tubular reactor concept for Reynolds number range of 10-100, Chem. Eng. Res. Des. 95, 22-33”. The technical data are listed in Table 1.

TABLE 1
Design Parameters of the CFI reactor for
continuously operated virus inactivation

| Design variable | Value | Unit |
|---|---|---|
| Inner diameter of tubing | 3.2 | Mm |
| Thickness of tubing wall | 1.6 | Mm |
| Frame diameter (coil) | 63 | Mm |
| Tubing distance within turn (minimum) | 6.4 | Mm |
| Number of turns per straight frame side | 9 | — |
| Number of straight frame sides | 20 | — |
| Number of 90° bends | 19 | — |
| Volumetric flow rate | 4.2 | ml min⁻¹ |
| Tubing length (Sanipure ® tube) | 40 | M |
| Hold-up volume of tubing | 322 | Ml |

The work which led to this application was funded in accordance with the “Bio.NRW: MoBiDiK—Modulare Bioproduktion—Disposable and Kontinuierlich” [Bio.NRW: MoBiDiK—modular bioproduction—disposable and continuous] grant agreement as part of the European Regional Development Fund (ERDF).

1. A method for the continuous elution of a biopharmaceutical, biological, macromolecular product from more than one chromatography column, comprising:
(a) providing a product stream via an inlet,
(b) simultaneously loading n front chromatography columns with the product stream in a loading zone with a loading time t_{B}, an outlet stream of the n front chromatography columns being simultaneously distributed to at least n-1 recovery chromatography columns in a recovery zone, where n is between 1 and 5, and
where the n front chromatography columns at a given time point have a varying loading time t_{B} between 0 and L₁, between L₁ and L₂, and up to between Lₙ₋₁ and Lₙ,
where each front chromatography column has a total loading time of Lₙ, wherein
periodically after a constant switch time t_{S} of Lₙ-Lₙ₋₁, the front chromatography column n comes out of the loading zone and a recovery chromatography column from the recovery zone enters the loading zone,
(c) washing the product-loaded column from step (b) with at least one wash buffer,
(d) eluting the product from the washed column from step (c) with an elution time t_{E}, which is ≥80% of the switch time t_{S}, and
that, over 80% of the total run time t_{G}, at least one chromatography column is continuously in elution step (d).

2. A method according to claim 1, wherein after elution (d), the eluted product is mixed by a homogenization (e), optionally by a recycling loop with a recycling tank, or by a recycling loop without a recycling tank, or by a single-use static mixer.

3. A method according to claim 1 wherein the method further comprises
(f) regenerating the eluted column from (d).

4. A method according to 1 wherein the front chromatography columns and the recovery chromatography columns bind product in accordance with the principle of affinity, via ionic interactions, via metal chelate binding, via hydrophobic interactions or via van der Waals forces, wherein the front chromatography columns and the recovery chromatography columns in the case of binding in accordance with the principle of affinity comprise a ligand optionally selected from the group consisting of protein A, protein G, protein L, IgM, IgG and a recombinant protein which is different from protein A, protein G, protein L, IgM and IgG and which has an affinity for the product.

5. A method according to 1 wherein the biopharmaceutical, biological macromolecular product is a protein, peptide or comprises a DNA or RNA, the protein or peptide being selected from the group consisting of monoclonal antibodies, polyclonal antibodies, recombinant proteins and protein vaccines, and the DNA or RNA being part of a DNA and/or RNA active ingredient or vaccine.

6. A method according to claim 1 wherein the pH of the eluted product from (d) is additionally adjusted by a pH adjuster, optionally before homogenization (e), or during homogenization (e).

7. A method according to claim 1 wherein the homogenized product from (e) is conducted through a defined residence-time passage, preferably optionally a coiled flow inverter (CFI).

8. A method according to claim 1 wherein the total run time t_{G} of the method is at least 4 hours, optionally at least 8 hours, optionally at least 12 hours, optionally at least 24 hours, optionally at least 48 hours, optionally at least 7 days, optionally at least 4 weeks, and optionally at least 8 weeks.

9. A method according to claim 1 wherein (a) to (d), optionally (a) to (f), are carried out in a microbe-reduced manner, wherein optionally all the used elements that come into contact with the product, optionally the front chromatography columns and the recovery chromatography columns, are subjected to microbe reduction by a suitable microbe-reduction method.

10. A method according to claim 9, wherein a suitable microbe-reduction method is selected from the group consisting of gamma irradiation, beta irradiation, autoclaving, ethylene oxide (ETO) treatment, ozone treatment (O₃), hydrogen peroxide treatment (H₂O₂) and steam-in-place (SIP) treatment.

11. A method according to claim 1, wherein all the liquids, gases and solids that are used in the method are subjected to microbe reduction, the microbe reduction optionally being achieved by a filtration through a filter having a pore size of optionally ≤0.45 μm, and that in-process sterilization is optionally not carried out during the method.

12. A method according to claim 1, wherein a degassing of all fluids which come onto the chromatography column is carried out before (b), the degassing being achieved optionally by at least one bubble trap and/or by at least one hydrophobic microfiltration membrane via vacuum and/or by treatment with ultrasound and/or by sparging with helium.
